(19) United States
(12) Patent Application Publication
(10) Pub. No.: US 2004/0028631 A1
(43) Pub. Date: Feb. 12, 2004

(54) Cosmetic formulations

(76) Inventor: Thomas Schwarz, Leichlingen (DE)

Correspondence Address: Robert Berliner, Fulbright & Jaworski, Twenty Ninth Floor, 865 South Figueroa, Los Angeles, CA, 90017-2571, US

(21) Appl. No.: 10/344,970

(22) PCT Filed: Aug. 16, 2001
(86) PCT No.: PCT/EP01/09446

(30) Foreign Application Priority Data
Aug. 18, 2000 (DE) 100 40 932.6
Aug. 22, 2000 (DE) 100 55 706.6

(51) Int. Cl. A61K 7/48

(52) U.S. Cl. 424/70.1

(57) ABSTRACT

The invention relates to the incorporation of di-sugar alcohol phosphates of C₃ to C₆ sugar alcohols and/or derivatives of said compounds, e.g. acids, salts, or esters, into cosmetic or dermatological formulations for protecting the skin against environmental influences.

The inventive low-molecular compounds of this type take part in the active cell protection of the cell's own free-radical scavengers, antioxidants, DNA, cell membranes and other cell compartments, protecting them against harmful environmental influences, e.g. UV radiation, IR radiation and environmental stress (thermal, chemical and physical).

The compatible solution can act as a co-solvent and intensify the penetration of other active ingredients added to the cosmetic formulation, not only to stabilize said ingredients in the formulation, but also to actively transport them into deeper dermal layers.

The invention relates to cosmetic and dermatological formulations, preparations or products.

### STATE OF THE ART

Protection Against UV and IR Radiation

Today's sun-protective agents comprise two classes of UV blocking filters. Physically active UV blocking filters consist of inorganic compounds (e.g. titanium dioxide) that reflect the light rays before hitting the skin's surface. Chemically acting organic molecules absorb the UV radiation of such wave lengths as are responsible for the formation of erythema (sunburn) (primarily between 295 and 323 nm with a maximum at 308 nm).

For that reason, all previous sunscreen preparations must be seen as mere “sunburn diminishers” that only guard against or prevent the dramatic effect of skin damage easily evident to the human eye. However, it has already been reported in literature that the even more dangerous damage to the cell caused well before the development of erythema may involve massive DNA damage that may even give rise to the formation of melanoma.

Free Radical Scavengers and Antioxidants

In the field of cosmetics a multitude of free radical scavengers and antioxidants has been employed as additives of cosmetic preparations to protect against oxidative stress caused by environmental influences (UV radiation, smoke, chemical substances) as well as by cell-inherent activities. The broad range of agents embraces not only water-soluble free radical scavengers (e.g. ascorbic acid) acting in the liquid inner cell but also fat-soluble antioxidants (e.g. Retionol palmitate or α-tocopherol) that have an effect on the membrane structures. In this context it is rarely mentioned that when these agents are applied topically not more than 3% of them reach deeper skin layers while the rest remains on the skin surface without having any effect.

Substance Penetration into Human Skin; Co-Solvents

Offering protection against harmful environmental influences human skin consists of several layers (stratum corneum, subdermis, dermis, epidermis) forming a barrier practically impenetrable to foreign substances. Potential routes of penetration open to cosmetic agents are intercellular (only for small polar and non-polar substances by lateral diffusion through the highly organized intercellular lipid layers), intracellular or transfollicular (liposome technology). There is evidence, however, that even when employing the innovative liposome encapsulation in nano-particles cosmetic agents are only capable of penetrating the upper skin layers at an extremely low percentage (<3%) to reach their targeted location where they are intended to take beneficial effect. Larger molecules such as enzymes promising in terms of potential effectiveness (e.g. sericin, kollytin, glutathion-S-transferase or the groups of cytokines) can be introduced into living cells to a minute extent only due to steric hindrance, even by applying the most modern methods available.

In view of this problematic situation there is a demand for novel formulations of improved cosmetic effectiveness within the fields mentioned above.

According to the invention this objective is reached by formulations specified in claim 1. Formulations that are particularly suited for these purposes are the subject of the dependent claims.

Hitherto, no substances have been known in preventive sun cosmetics that actively support the cell-inherent repair mechanisms of the cell and in this way safeguard the cell “from within” against elevated UV radiation. As a result of the proven DNA protection and protein stabilizing effect the use of di-sugar alcohol phosphates of C₃ to C₆ sugar alcohols constitute a novel way of active skin protection against UV radiation in cosmetics.

Surprisingly, it has now been ascertained that IR radiation leads to damaging the skin cells thermally and causes denaturization of cell proteins and enzymes. In the cosmetics industry no agents are known that offer protection against cell stresses caused by elevated temperatures. Extremophile microorganisms protect against thermal stress by forming compatible solutes such as DIP or DGP. Therefore, this category of substances appears to be suited for effective use in this still new area of application.

The invention is based on the unexpected finding that di-sugar alcohol phosphates of C₃ to C₆ sugar alcohols, in particular the compatible solutes DIP and DGP and/or derivatives of said compounds, for example acids, salts or esters, exhibit far-reaching efficacy for use in cosmetic or dermatological formulations. The sugar alcohol residues of the di-sugar alcohol phosphates according to the invention are alcohols of C₃ to C₆ sugars and may of linear or cyclic, modified or non-modified type. In this case the two sugar alcohol residues of a di-sugar alcohol phosphate may each be similar or dissimilar.

Every tissue has an antioxidative potential (AOP) stemming from enzymatic and non-enzymatic antioxidants that in the unstressed cells keep the content of pro-oxidants below a limit that does not mean harm to the healthy cell. According to the present invention the cell-inherent antioxidative potential is promoted by the application of di-sugar alcohol phosphates of C₃ to C₆ sugar alcohols through their proven protein stabilizing effect. Especially effective are the substances of di-myo-inositol phosphate (DIP) and di-glycerol phosphate (DGP) identified as compatible solutes in extremophile microorganisms.

Since they originate from extremophilic microorganisms that by producing said compatible solutes protect themselves effectively against environmentally induced stress factors as UV radiation, heat, cold, dryness or osmotic or chemical stresses and, only in this way, are capable of surviving, especially the DIP and DGP categories of substances stand for a new and innovative approach to counteract detrimental influences acting on the human skin.

The cell's inherent repair mechanisms (DNA repair after UV damage, induction and stabilization of the chaperon proteins to newly form (partially) denaturized proteins and enzymes) can be effectively stabilized and activated “from within” (in vivo) for the first time by DIP and DGP containing formulations, preparations and products. DIP and DGP enable the antioxidative potential of the cell triggered by a protein and DNA damage through radiation, free radicals or lipid oxidation of the cell membranes to be maintained.

Due to the low molecular weight di-sugar alcohol phosphates according to the invention penetrating through all skin layers enter the interior of the target cell and—acting as co-solvents or penetration intensifiers—are thus capable of carrying along other agents of cosmetic formulations (e.g. proteins, enzymes, vitamins, antioxidants) bringing them to the desired destination.

As a result of their chemical structure di-sugar alcohol phosphates according to the invention, particularly DIP or DGP, are compatible with the majority of the customary cosmetic basic formulations and further adjustments to the desired and suitable form of application can be achieved by appropriately modifying the functional side chains. In this process and due to numerous hydroxy groups di-sugar alcohol phosphates feature moisturizing properties and are very pleasant to the skin.

Compatible solutes act as natural solvents in extremophilic microorganisms and stabilize enzymes, proteins and other cell constituents by restoring a native, globular state that not only protects proteins against proteolysis but moreover keeps them in a more compact and active condition. As co-solvents or penetration intensifiers for cosmetic agents DIP and DGP can be beneficially and broadly used for various applications particularly in view of a possible derivatization of individual functional groups.

Various recognized in-vivo and in-vitro test procedures applied attest to the positive influence of the compatible solutes DIP and DGP in cosmetic and dermatological formulations.

With the aid of cell cultures the effect of the di-sugar alcohol phosphates according to the invention, especially DIP and DGP and their derivatives on a variety of environmental influences, particularly UV and IR radiation, can be easily shown ‘in vitro’. The antioxidative potential of tissue can be detected by means of electron spin resonance measurements by determining the RPF (radical protection factor).

By making use of the non-invasive biophotonic method it is possible to directly monitor the influence an external treatment has in terms of temporary and structural changes of the skin due to the reaction of the living cells or the tissue to stress or healing processes.

Eventually, the porcine skin model and tests involving human skin were employed for the purpose of determining the behavior of substances when penetrating individual skin layers.

Results

The excellent properties of di-myo-inositol phosphate towards the stabilization of proteins and enzymes under various stress conditions and its structural affinity with the elementary components of cell membranes and the membranes of a variety of cell organellae render this compatible solute especially interesting for application in the field of cosmetics.

Proteins and peptides in the form of agents gain more and more importance also in cosmetics and dermatology. Examples of applications of this nature in the field of cosmetics are, for instance, collagen preparations, the use of cytokines in preparations having anti-inflammatory effects, the protection of so-called microprotein or repair enzymes of the skin.

The skin-compatible stabilization and preservation of the functionality of these preparations to counteract temperature denaturization and proteolysis is decisive for the success of their application. The functional stabilization of proteins and peptides within the preparations (storage, transport) and, furthermore, after the preparation has been applied to the skin (depot effect) is of major significance. Particularly on the skin many defense mechanisms are present (e.g. proteases) that are ‘hostile to proteins’ and may impede the success of a protein-based cosmetic application.

The outstanding properties of di-myo-inositol phosphate in the stabilization of proteins and enzymes could be shown by experiment under various stress conditions.

Denaturization processes and the aging processes that have a causal relationship to them can always be characterized by a function that depends on the factors of time and temperature. Therefore, a temperature-induced denaturization will not only take place at extraordinarily high temperatures but as well at temperatures on a physiological level. For that reason, any increase in temperature stability translates in a slowdown of denaturization and aging processes. Agents that in this manner offer protection against skin aging are of enormous interest to the cosmetics industry.

As has been shown in experiments di-myo-inositol phosphate is highly capable of binding water and exhibits great stability characterized by the pronounced inclination of the numerous hydroxygroups in the molecule to form hydrogen bridges. Moreover, the compound is thus of interest for the regulation and balancing of the skin's moisture characteristics. This is at least of additional advantage when the compound is used as a cosmetic agent.

Desiccation/dehydration processes impair and damage skin constituents irreversibly in that cells, membranes and proteins are destroyed. Both the resistance and the regenerative capability of the skin diminish. Furthermore, skin aging processes accelerate in the end. Basically, the desiccation process the skin is subjected to is identical to a freeze-drying process, only the pertinent stress conditions are much more severe. Based on the protective potential of di-myo-inositol phosphate in the field of freeze drying it can thus be plausibly deducted that di-myo-inositol phosphate in conjunction with drying processes may act not only as a moisturizing agent but also as an actively functioning protective substance.

Structural Formula of Di-Myo-Inositol Phosphate

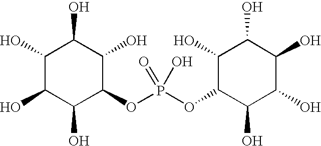

Application related examinations were conducted with di-glycerol phosphate (DGP) aimed at protecting model proteins. As a penetration intensifier di-glycerol phosphate moreover ensures that these macro-molecules are also transported through several skin layers until they have reached their target destination. Tests have clearly shown that di-glycerol phosphate has a by far better protective potential than, for example, glycerine, the preservation agent frequently employed in cosmetics.

Structural Formula of Di-Glycerol Phosphate

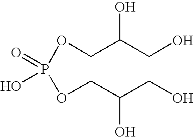

Subject of the invention is the use of one or more chemical compounds selected from the compounds of claim 1 in cosmetic and dermatological formulations. The invention, for example, relates to the manufacture of a cosmetic or dermatological formulation for the protection and stabilization of human skin cells as well as their organic constituents such as, for example, proteins, enzymes, membranes, nucleic acids or antioxidants. Preferred embodiments contain at least one of the compounds Ia and Ib as illustrated hereunder:

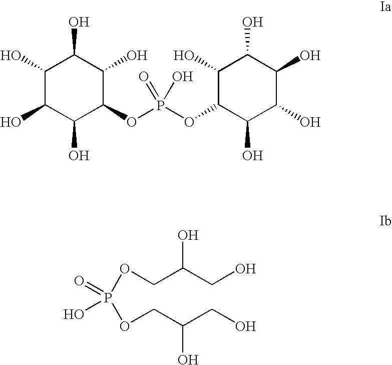

The invention embraces the use of physiologically compatible salts of the compounds according to claim 1, especially compounds Ia and Ib, the stereoisomeric forms of the these compounds and derivatives of these compounds (e.g. esters) for the use in cosmetic and dermatological formulations. As salts alkaline and alkaline-earth salts are suited, but in particular the Na and K salt. As esters all physiologically compatible esters can be employed.

Since they originate from extremophilic microorganisms that by producing novel compatible solutes such as di-inositol phosphate protect themselves effectively against environmentally induced stress factors as UV radiation, heat, radicals, cold, dryness or osmotic or chemical stresses and, only in this way, are capable of surviving, these substances stand for a completely new approach to the protection of organisms and their components from harmful environmental influences.

The cell's inherent repair mechanism (DNA repair after UV radiation damage, induction and stabilization of the chaperon proteins to newly form (partially) denaturized proteins and enzymes) can, for the first time, be stabilized and activated by DIP and DGP. DIP and DGP, for the first time, enable the antioxidative potential of the cell (protein and DNA damage through free radicals, lipid oxidation of the cell membranes) to be maintained.

Due to their low molecular weight DIP and DGP penetrating through several skin layers enter the interior of the target cell and—acting as co-solvents or penetration intensifiers—are thus capable of carrying along other agents of cosmetic formulations (e.g. proteins, enzymes, vitamins, antioxidants) bringing them to the desired destination.

As a result of their chemical structure especially DIP and DGP are compatible with the majority of the customary cosmetic basic formulations and further adjustments to the desired and suitable form of application can be achieved by appropriately modifying the functional side chains. In this process and due to numerous hydroxygroups di-inositol phosphate features moisturizing properties and is very pleasant to the skin.

Therefore, object of the invention is the use of di-sugar alcohol phosphates of C₃ to C₆ sugar alcohols and, in particular, DIP and/or DGP as additives and agents offering a completely novel functioning principle in cosmetic and dermatological preparations and products.

Compatible Solutes and Hyperthermophiles

Hyperthermophiles are rather extraordinary microorganisms because they grow optimally at temperatures (60-110° C.) that in the event of mesophilic (“normal”) organisms would lead to an extensive damage of cellular structures. In recent years comprehensive research efforts have been made to identify the biochemical components that bring about the remarkable thermal, chemical and physical stabilization of the cell structures. The focus of the research work in this context was on the isolation of thermally stable enzymes due to the fact that numerous enzymatically catalyzed industrial processes are carried out in extreme environments and suitable biocatalysts are required for these applications.

Although many enzymes from hyperthermophilic microorganisms are stable even under elevated temperatures this is not generally true for the cellular structures of thermophilic and hyperthermophilic organisms. The high temperature stability of cell structures is—to a remarkable extent—due to low-molecular organic substances (compatible solutes, “hypersolutes”) present in the intracellular environment. In recent years various new hypersolutes could be identified in hyperthermophilic microorganisms for the first time. In some cases it could be clearly shown that these compounds effectively contributed to the protection of cellular structures—first of all enzymes—against heat and dryness. bitop has developed technological solutions for the production of solutes from thermophilic microorganisms.

As interface and surface of the human body the skin is exposed to numerous external stresses. The human skin is an organ that consists of different, specialized cell types—the keratinozytes, melanozytes, Langerhans cells, Merkel cells as well as embedded sensory cells—and protects the body against external influences. In this context a distinction must be made between external physical, chemical and biological factors affecting the human skin. External physical influences are, inter alia, thermal and mechanical influences as well as the effects of radiation such as, for example, UV, VIS and IR radiation. External chemical influences particularly involve, inter alia, the effects of toxins, allergens and substances that attach to desoxyribonucleic acid. External biological influences mean the effects caused by foreign organisms and their metabolic products.

An excessive sun radiation leads to both acute skin damage such as for example sunburns and chronic changes such as, for example, skin aging or skin cancer. Sunburns (erythema solare) primarily develop when the skin is exposed to UV-B radiation. On the other hand, UV-A radiation by comparison has only a minor effect on the occurrence of sunburns. Sunburns of varying degree may develop ranging from a slight reddening to severe burns with blisters. Since these consequences will occur 4-6 h after exposure to radiation at the earliest, it will be too late to take countermeasures. Several sunburns—especially suffered during childhood—will increase the risk of skin cancer significantly. This is due to damage caused particularly to the nucleic acids of human skin cells and a faulty repair of the damaged desoxyribonucleic acid within the nucleus as well as possibly the immunosuppressive effect of UV radiation, i.e. a diminishing of the immune reaction as a result of the exposure to UV rays. An excessive exposure to UV-A and UV-B rays will increase the risk of a premature aging or light aging of the skin, for example in the form of structural changes of the connective tissue (actinic elastosis). An excessive exposure to UV-B rays is viewed as the main cause of chronic skin changes.

Due to changed recreational activities as, for instance, prolonged sunbathing or long-distance holidays in countries where the sun radiation is strong, dangers associated with skin cell damage due to UV exposure have increased severely in recent years which in turn entails a higher skin cancer risk. Moreover, the skin cancer risk has considerably increased lately as a result of the higher UV radiation present on the earth's surface on account of the stratospheric ozone depletion and also the higher life expectancy of mankind.

Aside from the still very topical problem of the lasting damage caused to cells due to UV radiation which by the use of customary sunscreens has hitherto been resolved only inadequately, the long underestimated influence of IR radiation damaging the skin cells thermally (thermal stress denaturizes cell proteins and enzymes) is being the subject of current discussions in research cycles. In the cosmetics industry no agents are known that offer protection against cell stresses caused by elevated temperatures.

Every tissue has an antioxidative potential (AOP) stemming from enzymatic and non-enzymatic antioxidants that in the unstressed cells keep the content of pro-oxidants below a limit that does not mean harm to the healthy cell. In the event these natural antioxidants become deactivated or denaturized the resistance as well as regenerative properties of the skin will decrease significantly.

Hitherto, only a few natural substances have been known in preventive cosmetics and dermatology that actively promote the cell-inherent repair mechanisms of the cell and in this manner safeguard the cell “from within” (in vivo) against, for example, elevated UV radiation, IR radiation, heat, free radicals, cold, dryness, osmotic stress or chemical stress. Therefore, the objective was to provide cosmetic and dermatological formulations whose application eliminates or at least alleviates the above described skin problems and which, in particular, are suited to protect and stabilize human skin cells and their organic components.

It is thus a general problem of cosmetics and dermatology that there are no clearly chemically defined and harmless agents and additives of natural origin available to satisfactorily resolve the above mentioned important objectives within the fields of application indicated. The present invention proposes a solution to this problem.

### INVENTION/BENEFICIAL EFFECTS OF THE INVENTION

Extremophilic microorganisms (thermophiles and hyperthermophiles) protect themselves against thermal stress by the formation of compatible solutes, in particular DIP and DGP. In the presence of these substances these microbial living organisms are capable of existing under extreme environmental conditions because these compounds serve to stabilize and protect the metabolism and the essential organic components against damage.

Surprisingly, it has now been ascertained that DIP and DGP also exhibit their stabilizing effects when applied to the human skin and thus must be seen as ideal agents and additives for cosmetic and dermatological preparations. It appears, furthermore, that these and similar effects can be produced by the substances belonging altogether to the category of di-sugar alcohol phosphates of C₃ to C₆ sugar alcohols. For that reason, di-sugar alcohol phosphates of C₃ to C₆ sugar alcohols represent a universally applicable and completely new protective principle in cosmetics and dermatology. According to the invention the above described problem is thus resolved by using one or several compounds in cosmetic and dermatological formulations. Additionally, the invention relates to the use of one or more of the above mentioned compounds, preferably compounds of formula Ia (di-inositol phosphate),

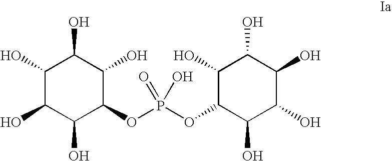

the physiologically compatible salts of compound Ia, the stereoisomeric forms of the compounds of formula Ia as well as of derivatives for the production of a cosmetic or dermatological formulation aimed at the protection and stabilization of human skin cells and their organic constituents such as, for example, proteins, enzymes, membranes, nucleic acids or antioxidants. Furthermore preferred are the compounds of formula Ib (di-glycerol phosphate),

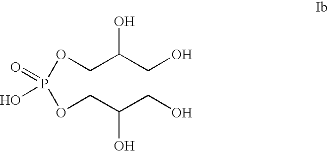

the physiologically compatible salts of compound Ib, the stereoisomeric forms of the compounds of formula Ib as well as of derivatives for the production of a cosmetic or dermatological formulation aimed at the protection and stabilization of human skin cells and their organic constituents such as, for example, proteins, enzymes, membranes, nucleic acids or antioxidants.

Moreover, the invention relates to the use of one or more of the above mentioned compounds, their physiologically compatible salts and/or stereoisomeric forms for the production of a cosmetic or dermatological formulation aimed at the protection and stabilization of human skin cells and their organic constituents such as, for example, proteins, enzymes, membranes, nucleic acids, antioxidants etc. to guard against physical, chemical and biological influences such as radiation (UV, IR, VIS rays), denaturizing substances, temperature or cold.

The present invention describes this protective principle for the first time. Possible fields of application are versatile and can only be described by way of examples within the framework of the invention. However, the invention shall not be limited in whatever way by giving or describing such examples.

Membranes constitute the natural barrier of cells and cell organellae at the surrounding medium. Membranes make sure that a constant inner environment prevails within the individual cells and cell constituents. Biological membranes are multi-component systems that usually consist of lipids, cholesterol and proteins. The exact composition of individual membranes varies to a greater or lesser extent depending the origin of the membranes. However, lipids and proteins are elementary constituents of membranes. Lipids can be subdivided into so-called “simple” lipids such as fats and waxes and “complex” lipids. As basic structural elements of membranes complex lipids such as glycerophospholipids (glycerine phosphatides) are most commonly found. The general structure of these membrane constituents is derived from sn-glycerine-3-phosphoric acid (see following figure).

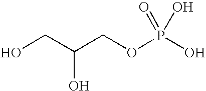

This is phosphoric acid diester in which the glycerine residue usually has condensed with saturated (R1) and unsaturated (R2) fatty acids. The phosphoric acid residue has additionally condensed with a component of alcoholic structure such as inositol or ethanolamine (R3)—(see following figure).

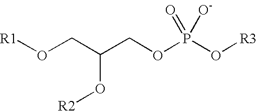

Due to its ambivalent structure comprising a hydrophilic phosphate residue and lipophilic fatty acid residues glycerophospholipids are capable of forming aggregates in aqueous solutions (e.g. bilayers or miccelles) in which only the hydrophilic phosphate residue is in contact with the aqueous phase. This structure is also found in the form of closed lamellar vesicles, the so-called liposomes.

DIP:

Phosphatidyl inositols represent a special category of phospholipids. They are nitrogen-free glycerophospholipids whose phosphoric acid ester group of the phosphatidic acid has been esterified with myo-inositol. In subcellular membranes they represent up to 10% of the entire lipids. Moreover, they take part in biochemical information transfer in some cases.

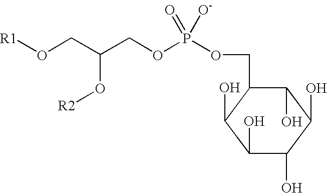

A comparison of the compatible solute di-myo-inositol phosphate (DIP) with the glycerophospholipids described above clearly attests to the structural relationship of the compounds. In the case of DIP the substituted glycerine residue of the lipid has been substituted by a myo-inositol residue.

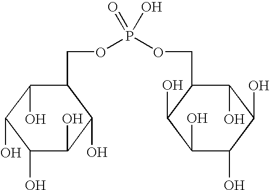

Di-myo-inositol phosphate exhibits the structural motives of classical stabilizers (many OH groups, phosphate) and thus combines in an ideal manner two important stabilization mechanisms:

Binding of hydrophilic protein domains via OH groups (simulation of water), prevention of hydrogen bridging within the protein structure and between proteins during drying and rehydration.

Presentation of a phosphate anion: Stabilization of protein side chains via ionic and van der Waals interaction. Furthermore, DIP can be distinguished by the following characteristics:

High chemical and physical stability

pH stability

Light stability

Colorless

Excellent solubility in water

No allergenic potential to be expected (substance from extremophiles)

Finally, the solubilizing effect of DIP substituting the water inside the cell must not be forgotten. Same as the other compatible solutes DIP is to be seen as the natural solvent of the cell. To actually take effect within the cell biogenic substances have to be solubilized.

| | Di-inositol phosphate |
|---|---|
| Molecular weight | 422.27 g/mol |
| Empirical formula | C₁₂H₂₃O₁₄P |
| Appearance | colorless solid |
| CAS number | 143491-08-1 |
| Optimum concentration | 250-400 mM |

With respect to the stabilizing effect of optically active DIP it is most important that the substance is present in the form of potassium salt.

Active Constiutuent of Liposome Structures

Phospholipids in the form of two-dimensional lamellae are capable of combining into lipid bubbles—so-called liposomes—into the center of which water-soluble components can be injected. Since liposomes due to their congeneric nature are able to merge with cell membranes agents can be introduced in this way into the cell with the help of liposomes. Stabilizing these lipid structures for storage and cosmetic application purposes is thus of major significance. On account of its structural affinity with lipids the use of the compatible solute of di-myo-inositol phosphate acting as a stabilizing agent is of interest here.

Thermoprotectors of Proteins

The excellent properties of di-myo-inositol phosphate towards the stabilization of proteins and enzymes under various stress conditions and its structural affinity with the elementary components of cell membranes and the membranes of a variety of cell organellae render this compatible solute especially interesting for application in the field of cosmetics.

Proteins and peptides in the form of agents gain more and more importance also in cosmetics and dermatology. Examples of applications of this nature in the field of cosmetics are, for instance, collagen preparations, the use of cytokines in preparations having anti-inflammatory effects, the protection of so-called microprotein or repair enzymes of the skin.

The skin-compatible stabilization and preservation of the functionality of these preparations to counteract temperature denaturization and proteolysis is decisive for the success of their application. The functional stabilization of proteins and peptides within the preparations (storage, transport) and, furthermore, after the preparation has been applied to the skin (depot effect) is of major significance. Particularly on the skin many defense mechanisms are present (e.g. proteases) that are ‘hostile to proteins’ and may impede the success of a protein-based cosmetic application.

Little is known at the present time about the effect of DIP in general. This mainly due to the fact that only very little of this compound is available. It has been shown by first experiments that in respect of the thermally induced denaturation of proteins the protective effect of DIP is excellent and in comparison with other compatible solutes is far and decidedly better.

Denaturization processes and the aging processes that have a causal relationship to them can always be characterized by a function that depends on the factors of time and temperature. Therefore, a temperature-induced denaturization will not only take place at extraordinarily high temperatures but as well at temperatures on a physiological level. For that reason, any increase in temperature stability translates in a slowdown of denaturization and aging processes. Agents that in this manner offer protection against skin aging are of enormous interest to the cosmetics industry.

DGP:

A comparison of the compatible solute di-glycerol phosphate (DGP) with the glycerophospholipids described above clearly attests to the pronounced structural relationship of the compounds. With R1, R2=H and R3=2,3 propanediol the most simple structure of a phospodiester with glycerine as alcohol component is present in DGP (see the following figure).

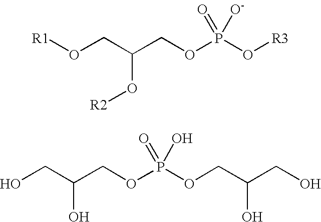

Based on today's knowledge there are two effects that primarily bring about the stabilization potential of DGP:

Binding of hydrophilic protein domains via OH groups (simulation of water), prevention of hydrogen bridging within the protein structure and between proteins during drying and rehydration.

Presentation of a phosphate anion: Stabilization of protein side chains via ionic and van der Waals interaction.

Furthermore, DGP can be distinguished by the following characteristics:

High chemical and physical stability

pH stability

Light stability

Colorless

Excellent solubility in water

High biocompatibility

Natural protective substance stemming from RI microorganisms

No allergenic potential to be expected (substance from extremophiles)

Finally, the solubilizing effect of DGP substituting the water inside the cell must not be forgotten. Same as the other compatible solutes DGP is to be seen as the natural solvent of the cell. To actually take effect within the cell biogenic substances have to be solubilized.

| | 1,1 Di-glycerol phosphate |
|---|---|
| Molecular weight | 246.15 |
| Empirical formula | C₆H₁₅O₈P |
| Appearance | colorless liquid, |
| | free acid |
| CAS number | 6418-92-4 |
| Optimum concentration | 100-200 mM |

Moisture Protection

The compound of DGP is highly capable of binding water characterized by the pronounced inclination of the numerous hydroxygroups in the molecule to form hydrogen bridges so that the compound is of interest for the regulation and balancing of the skin's moisture characteristics.

Protection of Liposomes and Membranes

Liposomes gain more and more importance as skin-penetrating vehicles, e.g. also for agents not solely used in the field of cosmetics. Liposomes are artificial lipid bilayers—artificial membranes—whose stability is limit, however. To enhance their storage and transportation properties there is a need for stabilizing liposome preparations to withstand, for example, temperature and osmotic stresses. This is the only way to make sure agents are effectively applied via liposomes.

Membranes constitute the natural barriers of cells and cell organellae at the surrounding medium. It is the membranes that make sure a constant inner environment prevails within the individual cells and cell constituents. Biological membranes are multi-component systems. The exact composition of individual membranes varies to a greater or lesser extent depending the origin of the membranes. However, lipids and proteins are elementary constituents of membranes.

Phospholipids in the form of two-dimensional lamellae are capable of combining into lipid bubbles—so-called liposomes—into the center of which water-soluble components can be injected. Stabilizing these lipid structures for storage and cosmetic application purposes is thus of major significance. On account of its structural affinity with lipids the use of the compatible solute of di-glycerol phosphate acting as a stabilizing agent is of interest here. This is furthermore backed by the fact that DGP as a constituent of lipid hydrolysates could be detected by NMR spectroscopy.

Protein Protection

Proteins and peptides in the form of agents gain more and more importance also in cosmetics. Examples of applications of this nature in the field of cosmetics are, for instance, collagen preparations, the use of cytokines in preparations having anti-inflammatory effects, the protection of so-called microprotein or regenerative enzymes of the skin.

The skin-compatible stabilization and preservation of the functionality of these preparations to counteract temperature denaturization and proteolysis is decisive for the success of their application. The functional stabilization of proteins and peptides within the preparations (storage, transport) and, furthermore, after the preparation has been applied to the skin (depot effect) is of major significance. Particularly on the skin many defense mechanisms are present (e.g. proteases) that are ‘hostile to proteins’ and may impede the success of a protein-based cosmetic application.

Use of the Invention in Trade and Industry

Using the invention in trade and industry encompasses the entire field of cosmetic and dermatological products as well as its application as research reagents in cosmetic and dermatological development work. Moreover, an application in trade and industry also involves the post-operative treatment of the skin (e.g. tending wounds in order to promote healing processes) and the addition of the compounds to plasters, masks and bandages as they are applied to the skin.

Further conceivable applications of compounds in cosmetic and dermatological fields are, for example:

Micro-encapsulation of cosmetic agents and additives

Stabilization of liposomes

Moisture dispensing

Free radical and UV protection

Protection against environmental pollutants and salts

Osmostress protectants

Ways to Implement the Invention Including Examples

The cosmetic and dermatological formulations are manufactured in that one or several compounds according to the invention, especially those of formulas la (di-inositol phosphate) or Ib (di-glycerol phosphate), their physiologically compatible salts, stereoisomeric forms or derivatives, if expedient with the aid of auxiliary and/or carrier substances, are appropriately dealt with so as to bring about a suitable formulation.

The auxiliary and carrier substances stem from the group of carrier agents, preservative agents and other customary auxiliary agents. The compounds of formula Ia (di-inositol phosphate), the physiologically compatible salts of the compounds according to the invention and their stereoisomeric forms as contained in cosmetic and dermatological formulations are for external application. They may for example be used in the form of solutions, suspensions, emulsions, pastes, ointments, gels, creams, lotions, powder, soaps, surfactant-containing cleaning preparations, oils and sprays. Moreover, customary carrier substances, auxiliary substances of any kind and, if thought expedient, further agents may be added to the formulations. Preferable auxiliary substances stem from the group of preservative agents, antioxidants, stabilizers, solutizers, vitamins, coloring agents and de-odorizers.

Aside from one or several compounds selected from the compounds according to the invention ointments, pastes, creams and gels may contain the customary carrier substances such as, for example, animal and vegetable fats, waxes, paraffins, starch, traganth, cellulose derivatives, polyethylene glycols, silicones, bentonites, silicic acid, talcum and zinc oxide or mixtures/blends of these substances.

Aside from one or several compounds according to the invention powders and sprays may contain in addition to the customary carrier substances the customary propellants, e.g. chlorofluorohydrocarbons, propane/butane or dimethyl ether.

Aside from one or several compounds according to the invention solutions and emulsions may additionally contain the customary carrier substances such as solvents, solutizers and emulgators or oils.

Aside from one or several compounds according to the invention suspensions may contain additional carrier substances such as, for example, water or ethanol.

Further forms of application are, for instance, soaps, surfactant-containing cleansing agents, face and body oils, lipsticks, lip-care sticks, mascara, eyeliners, eye shadowing, rouge, powder, emulsion and wax make-up, as well as sunscreen, pre-sun and after-sun preparations.

The proportion of the compounds according to the invention, their physiologically compatible salts, stereoisomeric forms in the cosmetic and dermatological formulations preferably amounts to 0.0001 to 50% by weight, especially preferred is 0.001 to 10% by weight based on the entire cosmetic formulation.

The cell-inherent antioxidative potential is promoted by the application of DIP and DGP, for example, through its proven stabilizing effect to prevent thermal protein denaturation and deactivation. In this way, an impairment as a result of free radicals is counteracted. As a result of the proven DNA protection of compatible solutes and their protein stabilizing effect the application of DIP or DGP according to the invention is to be regarded as a novel type of active sun protection (e.g. UV and IR radiation) in cosmetics and dermatology.

The following examples are meant to provide elucidation on the present invention but shall by no means limit its scope.

### EXAMPLE 1

O/W Lotion

According to the invention a di-inositol-phosphate-containing lotion (O/W) is made from the following components:

| Components | Percentage by Weight (% w/v) |
|---|---|
| Paraffin oil (DAB 9) | 8.00 |
| Isopropyl palmitate | 3.00 |
| Petrofatum | 4.00 |
| Cetylstearyl alcohol | 2.00 |
| PEG 40 castor oil | 0.50 |
| Sodium cetylstearyl sulfate | 0.50 |
| Sodium carbomer | 0.40 |
| Di-inositol phosphate | 0.50 |
| Glycerine | 3.00 |
| α-Tocopherol | 0.20 |
| Octylmethooxycinnamate | 5.00 |
| Butylmethoxydibenzoylmethane | 1.00 |
| Preservative agents, coloring matter, | q.s. |
| perfume | |
| Water | Ad 100.00 |

As preservative agent 0.05% propylhydroxybenzoate or 0.15% methyl-4-hydroxybenzoate may be used.

### EXAMPLE 2

O/W Cream

According to the invention a di-inositol-phosphate-containing cream (O/W) is made from the following components:

| Components | Percentage by Weight (% w/v) |
|---|---|
| Paraffin oil (DAB 9) | 7.00 |
| Avocado oil | 3.00 |
| Glycerylmonostearate | 4.00 |
| Di-inositol phosphate | 0.50 |
| Titanium dioxide | 1.00 |
| Sodium lactate | 3.00 |
| Glycerine | 3.00 |
| Preservative agents, coloring | q.s. |
| matter, perfume | |
| Water | Ad 100.00 |

As preservative agent 0.05% propylhydroxybenzoate or 0.15% methyl4-hydroxybenzoate may be used.

### EXAMPLE 3

Liposome-Containing Gel

According to the invention a liposome-containing gel that contains di-inositol phosphate is made from the following components:

| Components | Percentage by Weight (% w/v) |
|---|---|
| Lecithin | 6.00 |
| Shea butter | 3.00 |
| Di-inositol phosphate | 0.50 |
| á-Tocopherol | 0.20 |
| Biotin | 0.08 |
| Sodium citrate | 0.50 |
| Glycin | 0.20 |
| Urea | 0.20 |
| Sodium PCA | 0.50 |
| Hydrolized collagen | 2.00 |
| Xanthan gum | 1.40 |
| Sorbitol | 3.00 |
| Preservative agents, coloring | q.s. |
| matter, perfume | |
| Water | ad 100.00 |

As preservative agent 0.05% propylhydroxybenzoate or 0.15% methyl4-hydroxybenzoate may be used.

### EXAMPLE 4

Gel

According to the invention a di-inositol-phosphate-containing gel (O/W) is made from the following components:

| Components | Percentage by Weight (% w/v) |
|---|---|
| Carbopol | 2.00 |
| Triethanolamine | 3.00 |
| Di-inositol phosphate | 0.50 |
| α-Tocopherylacetate | 0.20 |
| Polyoxyethylene sorbitan fatty acid | 0.50 |
| ester (Tween 20) | |
| Glycerine | 2.00 |
| Sodium PCA | 0.50 |
| Hydrolized collagen | 2.00 |
| Preservative agents, coloring | q.s. |
| matter, perfume | |
| Water | ad 100.00 |

As preservative agent 0.05% propylhydroxybenzoate or 0.15% methyl4-hydroxybenzoate may be used.

### EXAMPLE 5

Sunscreen Emulsion

According to the invention a di-inositol-phosphate-containing sunscreen emulsion is made from the following components:

| Components | Percentage by Weight (% w/v) |
|---|---|
| Cyclomethicone | 2.00 |
| Cetyldimethicone copolyol | 0.20 |
| PEG 22 dodecyl copolymer | 3.00 |
| Paraffin oil (DAB 9) | 2.00 |
| Caprylic acid/Caprinic acid | 5.80 |
| triglyceride | |
| Octylmethoxycinnamate | 5.80 |
| Butyl methoxy dibenzoylmethane | 4.00 |
| α-Tocopherylacetate | 0.50 |
| ZnSO4 | 0.70 |
| Na3IIEDTA | 0.30 |
| Di-inositol phosphate | 0.50 |
| Preservative agents, coloring | q.s. |
| matter, perfume | |
| Water | ad 100.00 |

As preservative agent 0.05% propylhydroxybenzoate or 0.15% methyl-4-hydroxybenzoate may be used.

### EXAMPLE 6

Sun Lotion

According to the invention an emulgator-free di-inositol-phosphate-containing sunscreen lotion SPF 30 (W/O) is made from the following components:

| Components | Percentage by Weight (% w/v) |
|---|---|
| Caprylic acid/Caprinic acid | 30.00 |
| triglyceride | |
| Uvinul T150 | 4.0 |
| Eusolex 6300 | 2.00 |
| Neo Heliopan OS | 5.00 |
| Parsol 1789 | 2.00 |
| Eusolex T2000 | 4.00 |
| Aerosil R972 | 2.00 |
| Zinc oxide | 2.5 |
| Natrosol Plus 330CS | 0.5 |
| Glycerine | 10.0 |
| Di-inositol phosphate | 0.50 |
| Preservative agents, coloring | q.s. |
| matter, perfume | |
| Water | ad 100.00 |

As preservative agent 0.05% propylhydroxybenzoate or 0.15% methyl-4-hydroxybenzoate may be used.

### EXAMPLE 7

Hair Tonic

According to the invention a di-inositol-phosphate-containing hair tonic is made from the following components:

| Components | Percentage by Weight (% w/v) |
|---|---|
| Ethanol | 40.00 |
| Diisopropyladipate | 0.10 |
| α-Tocopherylacetate | 0.50 |
| Di-inositol phosphate | 0.50 |
| Preservative agents, coloring | q.s. |
| matter, perfume | |
| Water | ad 100.00 |

As preservative agent 0.05% propylhydroxybenzoate or 0.15% methyl-4-hydroxybenzoate may be used.

### EXAMPLE 8

Spray Formulation

According to the invention a di-inositol-phosphate-containing spray formulation is made from the following components:

| Components | Percentage by Weight (% w/v) |
|---|---|
| Ethanol | 28.2 |
| α-Tocopherylacetate | 0.10 |
| Di-inositol phosphate | 0.50 |
| Preservative agents, coloring | q.s. |
| matter, perfume | |
| Propane/butane 25/75 | Ad 100.00 |

As preservative agent 0.05% propylhydroxybenzoate or 0.15% methyl4-hydroxybenzoate may be used.

### EXAMPLE 9

O/W Lotion

According to the invention a di-glycerol-phosphate-containing lotion (O/W) is made from the following components:

| Components | Percentage by Weight (% w/v) |
|---|---|
| Paraffin oil (DAB 9) | 8.00 |
| Isopropyl palmitate | 3.00 |
| Petrofatum | 4.00 |
| Cetylstearyl alcohol | 2.00 |
| PEG 40 castor oil | 0.50 |
| Sodium cetylstearyl sulfate | 0.50 |
| Sodium carbomer | 0.40 |
| Di-glycerol phosphate | 0.50 |
| Glycerine | 3.00 |
| α-tocopherol | 0.20 |
| Octylmethooxycinnamate | 5.00 |
| Butylmethoxydibenzoylmethane | 1.00 |
| Preservative agents, coloring | q.s. |
| matter, perfume | |
| Water | Ad 100.00 |

As preservative agent 0.05% propylhydroxybenzoate or 0.15% methyl-4-hydroxybenzoate may be used.

### EXAMPLE 10

O/W Cream

According to the invention a di-glycerol-phosphate-containing cream (O/W) is made from the following components:

| Components | Percentage by Weight (% w/v) |
|---|---|
| Paraffin oil (DAB 9) | 7.00 |
| Avocado oil | 3.00 |
| Glycerylmonostearate | 4.00 |
| Di-glycerol phosphate | 0.50 |
| Titanium dioxide | 1.00 |
| Sodium lactate | 3.00 |
| Glycerine | 3.00 |
| Preservative agents, coloring | q.s. |
| matter, perfume | |
| Water | Ad 100.00 |

As preservative agent 0.05% propylhydroxybenzoate or 0.15% methyl-4-hydroxybenzoate may be used.

### EXAMPLE 11

Liposome-containing Gel

According to the invention a liposome-containing gel that contains di-glycerol phosphate is made from the following components:

| Components | Percentage by Weight (% w/v) |
|---|---|
| Lecithin | 6.00 |
| Shea butter | 3.00 |
| Di-glycerol phosphate | 0.50 |
| α-tocopherol | 0.20 |
| Biotin | 0.08 |
| Sodium citrate | 0.50 |
| Glycin | 0.20 |
| Urea | 0.20 |
| Sodium PCA | 0.50 |
| Hydrolized collagen | 2.00 |
| Xanthan gum | 1.40 |
| Sorbitol | 3.00 |
| Preservative agents, coloring | q.s. |
| matter, perfume | |
| Water | ad 100.00 |

As preservative agent 0.05% propylhydroxybenzoate or 0.15% methyl-4-hydroxybenzoate may be used.

### EXAMPLE 12

Gel

According to the invention a di-glycerol-phosphate-containing gel (O/W) is made from the following components:

| Components | Percentage by Weight (% w/v) |
|---|---|
| Carbopol | 2.00 |
| Triethanolamine | 3.00 |
| Di-glycerol phosphate | 0.50 |
| α-Tocopherylacetate | 0.20 |
| Polyoxyethylene sorbitan fatty acid | 0.50 |
| ester (Tween 20) | |
| Glycerine | 2.00 |
| Sodium PCA | 0.50 |
| Hydrolized collagen | 2.00 |
| Preservative agents, coloring matter, | q.s. |
| perfume | |
| Water | ad 100.00 |

As preservative agent 0.05% propylhydroxybenzoate or 0.15% methyl4-hydroxybenzoate may be used.

### EXAMPLE 13

Sunscreen Emulsion

According to the invention a di-glycerol-phosphate-containing sunscreen emulsion is made from the following components:

| Components | Percentage by Weight (% w/v) |
|---|---|
| Cyclomethicone | 2.00 |
| Cetyldimethicone copolyol | 0.20 |
| PEG 22 dodecyl copolymer | 3.00 |
| Paraffin oil (DAB 9) | 2.00 |
| Caprylic acid/Caprinic acid | 5.80 |
| triglyceride | |
| Octylmethoxycinnamate | 5.80 |
| Butyl methoxy dibenzoylmethane | 4.00 |
| α-Tocopherylacetate | 0.50 |
| ZnSO4 | 0.70 |
| Na3IIEDTA | 0.30 |
| Di-glycerol phosphate | 0.50 |
| Preservative agents, coloring | q.s. |
| matter, perfume | |
| Water | ad 100.00 |

As preservative agent 0.05% propylhydroxybenzoate or 0.15% methyl4-hydroxybenzoate may be used.

### EXAMPLE 14

Sun Lotion

According to the invention an emulgator-free di-glycerol-phosphate-containing sunscreen lotion SPF 30 (W/O) is made from the following components:

| Components | Percentage by Weight (% w/v) |
|---|---|
| Caprylic acid/Caprinic acid | 30.00 |
| triglyceride | |
| Uvinul T150 | 4.0 |
| Eusolex 6300 | 2.00 |
| Neo Heliopan OS | 5.00 |
| Parsol 1789 | 2.00 |
| Eusolex T2000 | 4.00 |
| Aerosil R972 | 2.00 |
| Zinc oxide | 2.5 |
| Natrosol Plus 330CS | 0.5 |
| Glycerine | 10.0 |
| Di-glycerol phosphate | 0.50 |
| Preservative agents, coloring | q.s. |
| matter, perfume | |
| Water | ad 100.00 |

As preservative agent 0.05% propylhydroxybenzoate or 0.15% methyl-4-hydroxybenzoate may be used.

### EXAMPLE 15

Hair Tonic

According to the invention a di-glycerol-phosphate-containing hair tonic is made from the following components:

| Components | Percentage by Weight (% w/v) |
|---|---|
| Ethanol | 40.00 |
| Diisopropyladipate | 0.10 |
| α-Tocopherylacetate | 0.50 |
| Di-glycerol phosphate | 0.50 |
| Preservative agents, coloring | q.s. |
| matter, perfume | |
| Water | ad 100.00 |

As preservative agent 0.05% propylhydroxybenzoate or 0.15% methyl-4-hydroxybenzoate may be used.

### EXAMPLE 16

Spray Formulation

According to the invention a di-glycerol-phosphate-containing spray formulation is made from the following components:

| Components | Percentage by Weight (% w/v) |
|---|---|
| Ethanol | 28.2 |
| α-Tocopherylacetate | 0.10 |
| Di-glycerol phosphate | 0.50 |
| Preservative agents, coloring | q.s. |
| matter, perfume | |
| Propane/butane 25/75 | Ad 100.00 |

As preservative agent 0.05% propylhydroxybenzoate or 0.15% methyl4-hydroxybenzoate may be used.

1. Cosmetic or dermatological formulation, preparation or product containing at least one di-sugar alcohol phosphate of C₃ to C₆ sugar alcohols, their physiologically compatible salts, stereoisomeric form or derivatives.

2. Cosmetic or dermatological topical formulation, preparation or product according to claim 1 containing at least one of the compounds of formula Ia, the physiologically compatible salts of the compounds of formula Ia, the stereoisomeric forms of the compounds of formula Ia or the derivatives of the compounds of formula Ia.

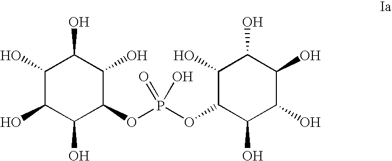

3. Cosmetic or dermatological topical formulation, preparation or product according to claim 1 containing at least one of the compounds of formula Ib, the physiologically compatible salts of the compounds of formula Ib, the stereoisomeric forms of the compounds of formula Ib or the derivatives of the compounds of formula Ib.

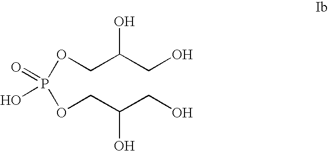

4. Formulation according to any one of claims 1 to 3, characterized in that the compounds, their physiologically compatible salts, stereoisomeric forms and/or derivatives are present in concentrations of 0.01 to 50% by weight, preferably 0.05 to 10% by weight, in particular 0.1 to 5% by weight based on the total weight of the formulation, preparation or product.

5. Formulations according to claims 1 and 2 for the protection and stabilization of human skin cells.

6. Formulation according to any one of the claims 1 to 4, characterized in that the nucleic acids of human skin cells are stabilized and protected against physical, chemical and biological influences.

7. Formulation according to any one of the claims 1 to 4, characterized in that the nucleic acids of human skin cells are protected against UV radiation and IR radiation.

8. Formulation according to any one of the claims 1 to 4, characterized in that the nucleic acids of human skin cells are stabilized and protected against denaturizing substances.

9. Formulation according to any one of the claims 1 to 4, characterized in that the nucleic acids of human skin cells are stabilized and/or protected against enzymes.

10. Formulation according to any one of the claims 1 to 4, characterized in that the human skin cells are stabilized and protected against viruses, viroids and prions.

11. Formulation according to any one of the claims 1 to 4, characterized in that human skin cells are stabilized and protected against low-infective agents.

12. Formulation according to any one of the claims 1 to 4 for the tending and/or prophylaxis of dry and/or scaly skin.

13. Formulation according to any one of the claims 1 to 4 for the protection of the human skin against dryness and/or elevated salt concentrations (higher than 1% w/w).

14. Formulation according to any one of the claims 1 to 4 for the protection of cells, proteins, protein complexes, lipoproteins, ribosomes and/or biomembranes of the human skin.

15. Formulation according to any one of the claims 1 to 4 for the protection of the microflora of the human skin.

16. Formulation according to any one of the claims 1 to 4 for the stabilization of the natural skin barrier.

17. Formulation according to any one of the claims 1 to 4 as water-retaining (water-binding) substance.

18. Formulation according to one of the claims 1 to 4 as free radical scavengers and/or antioxidants as well as its use for the treatment of and/or prophylaxis for skin aging caused by oxidative stress and of inflammatory reactions.

19. Formulation according to any one of the claims 1 to 4 for enhancing and accelerating the immune reaction of human skin cells for the protection of cells, proteins and/or biomembranes of human skin exposed to physical, chemical and biological stress.

20. Formulation according to any one of the claims 1 to 4, characterized in that the cosmetic or dermatological formulation contains one or several UV filters.

21. Formulation according to one of the claims 1 to 4, characterized in that the cosmetic or dermatological formulation contains one or several substances selected from enzymes, vitamins and vitamin derivatives.

22. Formulation according to any one of the claims 1 to 4, characterized in that the nucleic acids of the cells of the epidermis are protected and stabilized.

23. Use of one of the compounds of of claims 1 to 3, their physiologically compatible salts, stereoisomeric forms and/or derivatives or one of the formulations of claims 4 to 22, possibly together with one or several additional agents and additives for the external application in the form of a solution, a suspension, an emulsion, a paste, an ointment, a gel, a cream, a lotion, a powder, a soap, a surfactant-containing cleansing agent, an oil, a lipstick, a lip-care stick, a mascara, an eyeliners, of eye shadowing, rouge, a powder, emulsion and wax make-up, a sunscreen, pre-sun and after-sun preparation, a hair tonic, a plaster, a bandage or spray.

24. Cosmetic formulation containing one or several compounds selected from the compounds of claims 1 to 3, their physiologically compatible salts, stereoisomeric forms and/or derivatives.

25. Dermatological formulation containing one or several compounds selected from the compounds of claims 1 to 3, their physiologically compatible salts, stereoisomeric forms and/or derivatives.

26. Cosmetic formulation according to claim 24, characterized in that it is provided in the form of a solution, a suspension, an emulsion, a paste, an ointment, a gel, a cream, a lotion, a powder, a soap, a surfactant-containing cleansing agent, an oil, a lipstick, a lip-care stick, a mascara, an eyeliners, of eye shadowing, rouge, a powder, emulsion and wax make-up, a sunscreen, pre-sun and after-sun preparation, a hair tonic, a plaster, a bandage or spray.

27. Dermatological formulation according to claim 25, characterized in that it is provided in the form of a solution, a suspension, an emulsion, a paste, an ointment, a gel, a cream, a lotion, a powder, a soap, a surfactant-containing cleansing agent, an oil, a lipstick, a lip-care stick, a mascara, an eyeliners, of eye shadowing, rouge, a powder, emulsion and wax make-up, a sunscreen, pre-sun and after-sun preparation, a hair tonic, a plaster, a bandage or spray.
